(19) United States
(12) Patent Application Publication
(10) Pub. No.: US 2007/0282400 A1
(43) Pub. Date: Dec. 6, 2007

(54) Combination medical therapy device that integrates: electrical stimulation, light therapy, heat, pressure, and vibration

(76) Inventor: Kendall Jay Gorham, Beaverton, OR (US)

Correspondence Address: Kendall Gorham, 1504B Capstone Ct., Beaverton, OR, 97007, US

(21) Appl. No.: 11/445,653

(22) Filed: Jun. 2, 2006

(51) Int. Cl. A61N 5/06; A61F 7/00; A61N 1/00; A61H 1/00

(52) U.S. Cl. 607/88; 607/89; 607/96; 607/151; 601/15

(57) ABSTRACT

A portable combination medical therapy device that integrates the collective beneficial characteristics of five separate and distinct therapeutic pain relieving, relaxing, stimulating and healing modalities. The device comprises an applicator head that contains: at least two electrically conducting electrodes, a vibration mechanism, light mechanism, heat mechanism and is functionally designed to enable the operator to apply varying pressure and movement to the tissue being treated. The beneficial healing, relaxing and pain relieving results are enhanced because of the convenience and synergistic effect that the portable multiple healing modalities provide.

### CROSS-REFERENCE TO RELATED APPLICATIONS

Not Applicable

### STATEMENT REGARDING FEDERALLY SPONSORED RESEARCH AND DEVELOPMENT

Not applicable.

### REFERENCE TO SEQUENCE LISTING, A TABLE, OR COMPUTER PROGRAM LISTING COMPACT DISK APPENDIX

Not applicable

### BACKGROUND OF THE INVENTION

1. Technical Field

This invention relates to the medical fields of pain relief, healing, and facial rejuvenation. Specifically: a combination medical therapy device that integrates the separate healing modalities of electrical stimulation, light therapy, heat, vibration, and pressure.

2. Description of Relevant Art

| | | |
|---|---|---|
| 4,989,605 | February 1991 | Rossen |
| 5,304,207 | April 1994 | Stromer |
| 5,607461 | March 1997 | Lathrop |
| 6,013,096 | January 2000 | Tucek |
| 6,249,706 | June 2001 | Sabota |
| 6,267,779 | July 2001 | Gerdes |
| 6,413,267 | July 2002 | Dumoulin-White |
| 6,702,837 | March 2004 | Gutwein |
| 6,746,473 | June 2004 | Shanks |
| 6,896,693 | May 2005 | Sullivan |
| 5,358,503 | October 1994 | Bertwell |
| 6,187,029 | February 2001 | Shapiro |

All of the prior art inventions cited have significant inherent differences when it comes to treatment devices for pain relief and healing. The following inventions are pertinent and related to that of the patent because they use at least one of the five treating modalities that is incorporated in the present invention and they are generally intended to treat pain. It is often beneficial to provide multiple treatments simultaneously with a single medical therapy device. There is no reason, suggestion, teaching, or motivation found in any prior art whereby a person of ordinary skill in the field of the invention would incorporate the combination of modalities. The combination of features are not taught or even mentioned by any prior art especially in their preferred embodiment description.

Listed are the differences that are non-obvious and thus distinguish the current invention with prior art (all U.S.) that are cited:

U.S. Pat. No. 4,989,605, issued on Feb. 5, 1991, Rossen fails to use the healing modalities of vibration, pressure, and light therapy.

U.S. Pat. No. 5,304,207, issued Apr. 19, 1994, Stromer fails to use the healing modalities of vibration, pressure, heat, microcurrent, and light therapy. He describes a TENS unit used for masking pain with a current flow between 10-90 milliamps. He describes his light as a focused beam providing a guiding light spot to which electrical stimuli will be applied, which uses less than only 1 milliwatt of power. This is not enough power to provide any light therapy effects. The light is only a single beam that can only be located in the center of the applicator head and the light is incapable of emitting radiant heat or emitting therapeutic wavelengths less than 600 nanometers. His electrical signal and light is always ON or OFF together.

U.S. Pat. No. 5,607,461, issued on Mar. 4, 2007, Lathrop fails to use the healing modalities of vibration, pressure, heat and light therapy. His apparatus is strictly used for providing electrical stimulation to a lesion.

U.S. Pat. No. 6,013,096, issued Jan. 11, 2000 Tucek fails to use the healing modalities of vibration, pressure, heat, and microcurrent and his protruding cables are cumbersome.

U.S. Pat. No. 6,249,706, issued Jun. 19, 2001, Sobota fails to use the healing modalities of vibration, pressure, microcurrent, heat and light therapy. His apparatus uses very high voltage of 50-500 volts to apply to tissue or bone.

U.S. Pat. No. 6,267,779, issued Jul. 31, 2001, Gerdes fails to use the healing modalities of vibration, pressure, heat and electrical current. His apparatus is not portable and very cumbersome.

U.S. Pat. No. 6,413,267, Dumoulin-White issued Jul. 2, 2002, fails to use the healing modalities of vibration, pressure, heat and electrical current. His device is not portable.

U.S. Pat. No. 6,702,837, issued Mar. 9, 2004, Gutwein fails to use the healing modalities of vibration, pressure, heat, and microcurrent. His light is infrared only and cannot emit therapeutic light below 840 nanometers.

U.S. Pat. No. 6,746,473, issued Jun. 8, 2004, Shanks fails to use the healing modalities of vibration, pressure, heat, and electrical current.

U.S. Pat. No. 6,896,693, issued May 24, 2005, Sullivan fails to use the healing modalities of electrical current, heat, vibration, and pressure. His invention is a non-portable casket-like chamber for laying an entire body into for overall light stimulation.

U.S. Pat. No. 5,358,503, issued. Oct. 25, 2004, Bertwell fails to use the healing modalities of electrical current, vibration, and pressure. His invention is not a portable hand-held device.

U.S. Pat. No. 6,187,029, issued Feb. 13, 2001, Shapiro fails to use the healing modalities of electrical current, vibration, and pressure. His applicator head surface area is not flat but concave.

As stated above, the five principal modalities used in this invention; light therapy, heat, electrical current, vibration and pressure (massage) all have shortcomings when used as a sole treatment method for pain or healing. However, when used in combination these five modalities have produced unexpected beneficial synergistic healing results.

3. Background of the Invention

It is generally an accepted practice to administer medical treatment by way of the individual modalities of light therapy, heat, electrical stimulation, vibration, and pressure (massage). Individually, these five remedies are well known and have been used for many years in the field of pain therapy, healing and facial rejuvenation. Each individual mode of pain relief has its benefits and deficiencies:

Electrical stimulation benefits: Electrical stimulation therapy represents a significant improvement in rapid pain control and acceleration of healing. Electrical current therapy is usually administered through conductive pads positioned so that current flows between them through the injured area. The key to the understanding and success of electrical current therapy lies within its ability to stimulate healing at the cellular level. Injury to the body disrupts its normal electrical activity; electrical current therapy produces electrical signals similar to those that naturally occur when the body is repairing damaged tissues. These machines work at the cellular level by creating a vehicle of electrical current to compensate for the diminished bioelectrical current available to injured tissue. This enhances the body's ability to transport nutrients to and wastes from the cells in the affected area.

Electrical stimulation deficiencies: Electrical stimulation equipment exists but suffers from a number of limitations well known in the art. For example, much of present day equipment is bulky or requires placement of needle electrodes or other separate electrodes taped to the body and connected by wires to the apparatus. TENS (transcutaneous electrical nerve stimulation) and high-voltage pulsed galvanic stimulators deliver amperage that exceeds the nerve firing threshold, resulting in sensation ranging from a gentle tingling to intense muscle throbbing which results in merely masking the pain, but does nothing to promote healing. The general intention of TENS therapy is being served when the pain is masked because the resulting sensation when applied, is more tolerant than the pain. Thus traditional TENS only works if the current is strong enough to mask the pain.

Light therapy benefits: Light therapy is used in the treatment of a broad range of conditions. Light therapy improves wound healing, improves acne, stimulates production of collagen, reduces edema, and relieves pain of various etiologies. In light therapy, red and infrared light have different effects on cells and molecules. Red (visible) light can produce chemical changes while infrared radiation generally produces physical changes in molecules. In spite of this, both result in clinical improvement. Visible light enhances cell proliferation through photochemical changes in the mitochondria, which then set in motion a chain of biological events that ultimately, affect cellular membranes. This, in turn, has an effect on messenger RNA synthesis, which ultimately leads to the observed enhancement of cell proliferation. Pores in membranes open and close to let ions, such as calcium, in and out of cells as a consequence of physical changes in the membrane pore molecules. Calcium ions act as intracellular messengers in many signal-transducing pathways. The cellular calcium ion concentration can be abruptly raised for signaling purposes by transiently opening calcium channels in the plasma or intracellular membranes. The catalytic activities of many enzymes are regulated by the calcium concentration. Since infrared radiation affects the physical state of molecules, they can affect the pore molecules directly. Thus, a similar effect on cell proliferation can occur whether the cells were irradiated with visible light at 633 nm or infrared at 830 nm. Specific types of molecules absorb specific wavelengths of light, both visible and infrared. Absorbed radiation produces specific biological effects in tissue, depending upon which types of molecules absorb the light. Each color or wavelength between 280-1500 nanometers produces light that has it's own benefit to healing and well-being.

Light therapy deficiencies: Light therapy exists but suffers from a number of limitations well known in the art. For example, much of present day equipment is bulky, uses light waves that use ineffective single wavelengths and lack the power to penetrate and stimulate appropriate biological tissues. Light therapy used as a single modality to treat injury has shown limited treatment results.

Heat therapy benefits: The heating of tissue is used to increased blood flow and to provide soothing relief of the tissue being treated.

Heat therapy deficiencies: Used as a single treatment modality, heat has proven to have incomplete beneficial results in the treatment of pain and injury.

Therapeutic vibration benefits: Therapeutic vibration relaxes tight muscles and stimulates circulation throughout the body, which results in relief of muscle tension and relaxation. Reflexology, which incorporates proper vibration stimulation that is applied to the soles of the feet, can have a significant healing effect on the entire body. When properly applied, vibration therapy gives you a feeling of well-being and relaxation.

Therapeutic vibration deficiencies: Therapeutic vibration when used as a single therapy modality is generally only moderately effective.

Pressure therapy (massage) benefits: Increases blood and lymphatic circulation, eliminates toxic waste from muscle areas, helps to cleanse the skin from dead cells, and creates a relaxing effect on the muscles and surrounding tissue. It helps to relief muscular aches, mostly back, neck and shoulders aches where most people accumulate tension because of overwork or bad posture. By increasing the blood flow in the muscles, the muscular oxygenation will be augmented allowing them to heal faster.

Pressure therapy deficiencies: Pressure (massage) used as a single therapy modality has limited results in relieving pain and promoting healing.

### BRIEF SUMMARY OF THE INVENTION

The present invention is a portable hand-held combination therapy device, which integrates multiple modalities of energy and offers the collective beneficial characteristics of five distinct and separate pain relieving and healing modalities. This invention is unique and unprecedented in the field of pain relief, relaxation and healing because it grants access to five treatment modalities simultaneously or selectively. The healing and pain relieving results are enhanced because of the synergistic effect that it provides. Although the invention is principally described herein for use with human patients or subjects, the invention is equally beneficial to livestock and domestic animals.

### BRIEF DESCRIPTION OF THE DRAWINGS

The present invention provides a unique portable hand-held combination therapy device for incorporating five separate healing treatment methods to enhance treatment, relaxation and curing of biological tissue. As used herein, the term “biological tissue” includes skin, muscle, bone, nerves, tendons, and other parts of a biological organism.

FIG. 1 is a view of the present device and a detailed view of the applicator head common plane that comes into contact with biological tissue that is being treated. The portion of the device that is not shown is the remainder of the handle 22, which can extend to any length that is desirable, usually about 12 inches.

FIG. 2 is a block diagram illustrating the components making up a typical electrical circuit according to the present invention.

### DETAILED DESCRIPTION OF THE INVENTION

A need exists for an improved medical device that more conveniently and effectively treats biological tissue in the areas of pain reduction, relaxation, healing, and facial rejuvenation. Often it is advantageous to treat a single injury with multiple treatment modalities so as to increase the effectiveness of the results. Since each therapy has its own treatment device, specifications, and benefits; treating a patient can require using multiple devices, each of varying convenience and effectiveness. Therefore, an object of the present invention is to provide a device that is capable of integrating a combination of multiple therapy modalities that can be used either simultaneously or selectively so as to achieve superior synergistic therapeutic results.

The present invention is configured as a portable hand-held combination medical therapy device, which integrates the collective beneficial modalities of energy from light therapy, heat, electrical stimulation, vibration and pressure. The device consists of an applicator head that contains at least two electrically induced electrodes, multiple lights of varying wavelengths, a heat mechanism, vibration mechanism, and ergonomically designed so the operator can apply varying movement and therapeutic pressure to the treatment area.

The present invention consists of the following preferred embodiments:

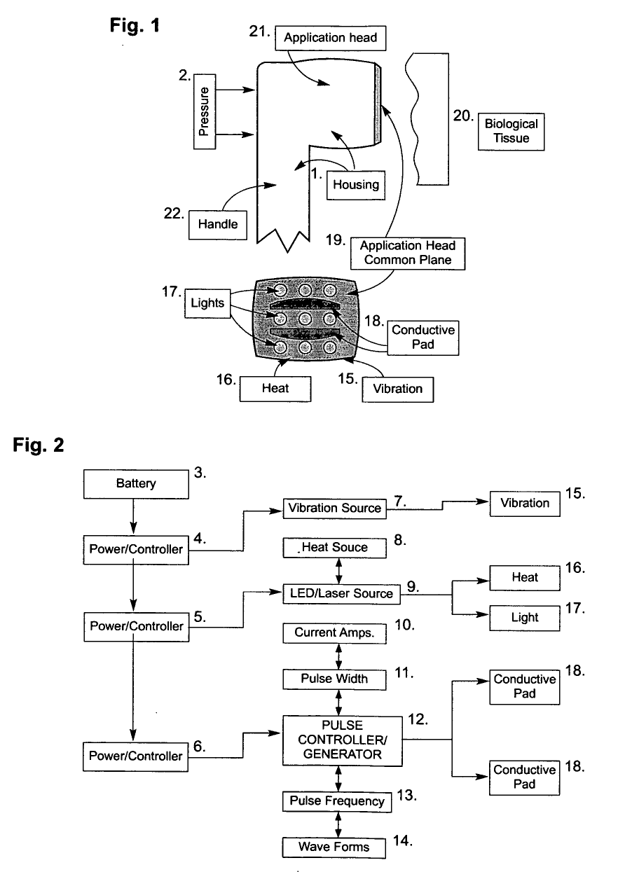

FIG. 1 shows a portable housing 1 which is functionally designed so that that the applicator head portion of the housing is protruding outward and angled approximately perpendicular to the handle portion. The housing 1 contains a handle 22, an applicator head 21, electrical circuitry and an energy source 3. The device is ergonomically designed and configured so that the operator can easily apply manual movement and/or stimulating pressure 2 to the applicator head common plane 19 when treating the surface area of biological tissue 20. Generally, the operator will supply constant movement of the applicator head in a circular or back and forth sweeping motion around the injury zone and has the option of pressing the applicator head with substantial force to initiate pressure on the muscles or biological tissue that is being treated. This may be critical in obtaining the necessary therapeutic results from the various modalities. Attached to the housing in a convenient location are: power/controller means for the vibration 4, lights and/or heat 5, and electrical generator 6. Also included in the housing may be a display screen indicating the various levels of output that is generated during treatment and possibly a timer that indicates treatment time (neither is shown).

The applicator head contains a vibration source 7, heat source 8, and light source 9 and a control mechanism 12 that allows the user to selectively adjust various output levels such as amperage 10, pulse width 11, pulse frequency 13, and wave forms 14. The applicator head common plane 20 (FIGS. 1 and 2) contains: two or more conductive pads 18 spaced apart at a predetermined distance for making surface contact with biological tissue being treated, and a combination of multiple laser and/or LED lights 17 that shine outward onto the treatment area. It is desirable that the conductive pads are slightly raised and protruding from the applicator head common plane to promote easier electrical contact to the biological tissue as it is being treated. The applicator head common plane outer surface is also capable of providing vibration 15 and heat 16 to biological tissue 20 that is being treated. Prior to treating with electrical stimulation it is preferred that a thin layer of conductive gel is applied to the area being treated to minimize contact resistance. All five treatment modalities can be used simultaneously or individually depending on the specific treatment requirements.

The specifications for the light are adjustable and have a wide range of variance because it is important to treat each patient with the appropriate stimulation. The therapeutic wavelengths may vary within the range of approximately 280-1500 nanometers and between 1 milliwatt to 2 watts or higher. The higher wattage is particularly important for deeper penetration requirements. Multiple wavelengths and power are important because they allow for various penetration levels and individualized treatment regimens.

The specifications for the heat source are variable. The heat source could be from a resister, heat sinks, radiant heat, or any other method that one skilled in the art would be able to create. The most effective heat temperature for healing and relaxation is usually between 95-120 degrees.

The specifications for the electrical output are as follows: pulse width 1-300 microseconds or higher; pulse frequency 1-250 hertz or higher; and amperage output level of 0-90 milliamps or higher. The waveform can vary significantly between square, rectangle, step etc. The waveform can also be programmed to fluctuate between various forms. The polarity output can be continuous or bi-phasic. Wide specification ranges are advantageous because of greater flexibility in treatment requirements. Biological tissue often responds better to varying the electrical output levels during treatment.

It is sometimes advantageous to enable multiple healing modalities simultaneously. A rapid synergistic healing effect sometimes occurs when multiple modalities are used in combination. The injured tissue responds to multiple stimulation modalities by relaxing muscles, increasing blood flow, and stimulating the cells and surrounding tissue. This combined stimulation assists in producing the desired biological changes necessary to enable the tissue to heal more rapidly.

While the present invention has been described for convenience of explanation in terms of particular structures and arrangements and circuits and materials, those of skill in the art will appreciate based on the description herein that many variations can be made without departing from the spirit of the present invention. Thus, it is intended to include these and such other variations as will occur to those of skill in the art based on this disclosure in the claims that follow.

What I claim as my invention is:

1. A combination medical therapy device, comprising:
a portable housing that contains an applicator head, electrical circuitry and an energy source;
a relatively flat rigid common plane portion of the applicator head that contains first and second electrodes which are exposed and spaced apart at a predetermined fixed distance;
an electrical signal generating mechanism supported by the housing and configured for emitting varying electrical signals to the electrodes;
a light emitting mechanism with wavelengths between 280-1500 nanometers and power output of each light between 1 milliwatt and 2 watts and supported by the applicator head to direct light waves substantially perpendicular to said common plane.

2. A device as defined in claim 1 wherein affixed to the applicator head is a heat source mechanism capable of producing heat of approximately 95-120 degrees to the said common plane of the applicator head;

3. A device as defined in claim 1 wherein affixed to the housing is a display screen.

4. A device as defined in claim 1 wherein exists a vibration mechanism supported by the applicator head to provide vibration to the said common plane.

5. A device defined in claim 1 wherein the electrical signal generating mechanism is capable of selectively adjusting output pulse signals of the pulse width from about 1 to 300 microseconds, the pulse frequency from about 1 to 250 Hz, and amperage output from about 5 microamperes to 90 milliamperes.

6. A device defined in claim 1 wherein it is ergonomically designed and configured so that the operator can impose substantial pressure and movement to the applicator head common plane by applying downward pressure to the opposite end of the applicator head.

7. A device as defined in claim 1 that includes a timer mechanism.

8. A combination medical therapy device, comprising:
a portable hand-held housing that contains an applicator head, electrical circuitry and an energy source;
first and second electrodes located on the applicator head within a relatively flat shaped common plane and spaced apart at a predetermined fixed distance;
an electrical signal generating mechanism supported by the housing and configured for emitting varying electrical signals to the electrodes;
a heat source mechanism capable of producing heat of approximately 95-120 degrees to the said common plane of the applicator head.

9. A device as defined in claim 8 wherein exists a light emitting mechanism with wavelengths configured between 280-1500 nanometers and power output of each light between 1 milliwatt and 2 watts and supported by the applicator head to direct light waves substantially perpendicular to said common plane;

10. A device as defined in claim 8 wherein exists a vibration mechanism supported by the applicator head.

11. A device defined in claim 8 wherein it is ergonomically designed and configured so that the operator can impose substantial pressure and movement to the applicator head common plane by applying downward pressure to the opposite end of the applicator head.

12. A device as defined in claim 8 wherein affixed to the housing is a display screen and an on/off means for independently controlling power generation to the electrodes, lights, heat, and the vibration mechanism.

13. A device as defined in claim 8 wherein the electrical signal generating mechanism is capable of selectively adjusting output waveforms.

14. A device defined in claim 8 wherein the electrical signal generating mechanism is capable of selectively adjusting output pulse signals of the pulse width from about 1 to 300 microseconds.

15. A device defined in claim 8 wherein the electrical signal generating mechanism is capable of selectively adjusting output pulse signals of the pulse frequency from about 1 to 300 Hz.

16. A device defined in claim 8 wherein the electrical signal generating mechanism is capable of selectively adjusting output of the current amperage from about 5 microamperes to 90 milliamperes.

17. A combination medical therapy device, comprising:
a portable hand-held housing that contains an applicator head, electrical circuitry and an energy source;
first and second electrodes located on the applicator head within a relatively flat shaped common plane and spaced apart at a predetermined fixed distance;
a heat source mechanism capable of producing heat of approximately 95-120 degrees to the said common plane of the applicator head;
a vibration mechanism supported by the applicator head.

18. A device defined in claim 17 wherein included is a light emitting mechanism supported by the applicator head to direct light substantially perpendicular to said common plane.

19. A device defined in claim 17 wherein the electrical signal generating mechanism is capable of selectively adjusting output waveforms.

20. A device defined in claim 17 wherein the electrical signal generating mechanism is capable of selectively adjusting output of the current amperage from about 5 microamperes to 90 milliamperes.
